(19) United States
(12) Patent Application Publication
(10) Pub. No.: US 2022/0031653 A1
(43) Pub. Date: Feb. 3, 2022

(54) Compounds for Treating Tauopathies and Restless Leg Syndrome and Methods of Using and Screening for Same

(71) Applicant: United States Government as Represented by the Department of Veterans Affairs, Washington, DC (US)

(72) Inventors: George Richard Uhl, Albuquerque, NM (US); Ian M. Henderson, Sandia Park, NM (US)

(21) Appl. No.: 17/390,497

(22) Filed: Jul. 30, 2021

Related U.S. Application Data
(60) Provisional application No. 63/059,038, filed on Jul. 30, 2020.

(51) Int. Cl. A61K 31/352; C12Q 1/42

(52) U.S. Cl. CPC A61K 31/352; G01N 2333/91205; C12Q 1/42

(57) ABSTRACT

Provided are compounds capable of enhancing the ability of receptor-type tyrosine-protein phosphatase delta (PTPRD) to dephosphorylate a kinase. Also disclosed is a method of treating a tauopathy or restless leg syndrome in a subject comprising administering to the subject an effective amount of a disclosed compound. Also disclosed are kits comprising the compounds together with instructions for treating a condition and/or a compound known for treating the condition. Finally, disclosed herein is a screening method suitable for identifying positive allosteric modulators of the ability of a receptor-type tyrosine-protein phosphatase delta (PTPRD) to dephosphorylate a kinase.

### CROSS-REFERENCE TO RELATED APPLICATIONS

This application claims priority to U.S. Provisional Application No. 63/059,038, filed Jul. 30, 2020, which is incorporated by reference in its entirety.

### REFERENCE TO SEQUENCE LISTING

The Sequence Listing submitted Jul. 30, 2021 as a text file named “37759.316U1_ST25.txt,” created on Jul. 24, 2020, and having a size of 11,000 bytes is hereby incorporated by reference pursuant to 37 C.F.R. § 1.52(e)(5).

### BACKGROUND

A misfolded and excessively phosphorylated version of “microtubule-associated protein tau,” or “tau,” is a major protein constituent of neurofibrillary tangles (NFTs), one of the two notable indicators of human Alzheimer's disease. Tau aggregates are also the primary pathological feature of various other neurodegenerative disorders collectively referred to as “tauopathies.” Tauopathies include, for example, Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, and progressive supranuclear palsy.

Understanding tau pathophysiology is important for developing therapies targeted at tauopathies and related conditions. Brains of individuals who die with Alzheimer's disease and other tauopathies often contain amyloid-rich senile plaques and NFTs rich in hyperphosphorylated, misfolded, aggregated tau. See E. E. Congdon, E. M. Sigurdsson, “Tau-targeting therapies for Alzheimer disease.” Nat. Rev. Neurol. 14, 399-415 (2018). Moreover, good correlations between cerebral cortical densities of NFTs and in vivo cognitive testing results link neurofibrillary pathology with Alzheimer's disease dementia. See P. Giannakopoulos, G. Gold, A. von Gunten, P. R. Hof, C. Bouras, “Pathological substrates of cognitive decline in Alzheimer's disease.” Front Neurol. Neurosci. 24, 20-29 (2009); G. K. Wilcock, M. M. Esiri, “Plaques, tangles and dementia. A quantitative study.” J. Neurol. Sci. 56, 343-356 (1982).

Pathogenic tau hyperphosphorylation is attributed to kinases that include the glycogen synthase kinases GSK3β and GSK3α, as well as CDK5, an atypical member of the cyclin dependent kinase gene family. See T. Kimura, K. Ishiguro, S. Hisanaga, “Physiological and pathological phosphorylation of tau by Cdk5.” Front Mol. Neurosci. 7, 65 (2014); A. Cavallini et al., “An unbiased approach to identifying tau kinases that phosphorylate tau at sites associated with Alzheimer disease.” J. Biol. Chem. 288, 23331-23347 (2013); E. Lauretti, O. Dincer, D. Pratico, “Glycogen synthase kinase-3 signaling in Alzheimer's disease.” Biochim. Biophys. Acta. Mol. Cell Res. 1867, 118664 (2020). Activities of these kinases can be regulated by their own phosphorylation. See R. Dhavan, L. H. Tsai, A decade of CDK5. Nat. Rev. Mol. Cell Biol. 2, 749-759 (2001); K. Hughes, E. Nikolakaki, S. E. Plyte, N. F. Totty, J. R. Woodgett, “Modulation of the glycogen synthase kinase-3 family by tyrosine phosphorylation.” EMBO J 12, 803-808 (1993); K. Shah, D. K. Lahiri, “Cdk5 activity in the brain—multiple paths of regulation.” J Cell Sci. 127, 2391-2400 (2014). GSK3β, GSK3a and CDK5 activities can be enhanced by phosphorylation of their tyrosines 216, 279 and 15, respectively. See, e.g., K. Hughes, E. Nikolakaki, S. E. Plyte, N. F. Totty, J. R. Woodgett, “Modulation of the glycogen synthase kinase-3 family by tyrosine phosphorylation.” EMBO J 12, 803-808 (1993).

Dephosphorylation of these tau-phosphorylating enzymes is also important. There are increased GSK3β, GSK3a and CDK5 activities or activating phosphorylation when cultured, expressing cells are treated with vanadate, a nonselective inhibitor of protein tyrosine phosphatases. See D. Simon et al., “Pharmacological inhibition of GSK-3 is not strictly correlated with a decrease in tyrosine phosphorylation of residues 216/279.” J Neurosci. Res. 86, 668-674 (2008); H. Kobayashi et al., “Phosphorylation of cyclin-dependent kinase 5 (Cdk5) at Tyr-15 is inhibited by Cdk5 activators and does not contribute to the activation of Cdk5.” J Biol Chem 289, 19627-19636 (2014). However, the specific tyrosine phosphatase(s) that dephosphorylate and thus downregulate GSK3β, GSK3α and/or CDK5 activities in the brain have not been reported. Understanding the dephosphorylation mechanisms of these kinases is needed before adequate therapies can be developed for the treatment of tauopathies.

Similarly, other addiction, locomotion, and sleep-related disorders can also have an association with tyrosine phosphatases, whether or not such disorders are associated with hyperphosphorylated tau or NFTs. Some studies show, for example, that there is an association between the expression of phosphatase genes in restless leg syndrome (RLS, also known as Willis-Ekbom disease). Accordingly, there exists a need in the art for both understanding tau pathophysiology and the role of phosphatase activity in other disorders such as RLS, as well as a need for identifying compounds useful to targeting such pathophysiologies and phosphatase activities. These needs and others are met by the following disclosure.

### SUMMARY

In one aspect, this disclosure relates to a method of treating a tauopathy or restless leg syndrome in a subject, comprising administering to the subject an effective amount of a compound represented by Formula (I):

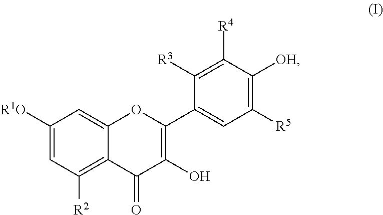

or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

In a further aspect, disclosed is a method of enhancing the ability of receptor-type tyrosine-protein phosphatase delta (PTPRD) to dephosphorylate a kinase, the method comprising contacting PTPRD with an effective amount of a compound represented by Formula (I):

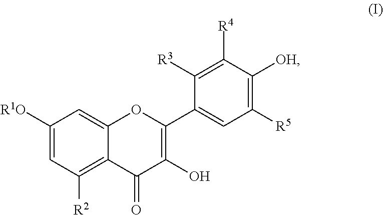

or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

In another aspect, disclosed is a kit comprising: (a) a compound represented by Formula (I) in an amount effective for treating a tauopathy or restless leg syndrome in a subject,

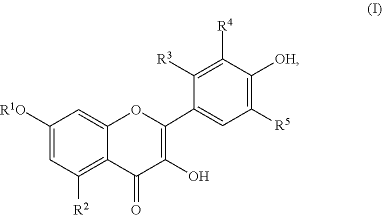

or a pharmaceutically acceptable salt thereof; wherein R¹ is hydrogen or —CH₃; R², R³, and R⁵ are each independently hydrogen or —OH; and R⁴ is hydrogen, —OH, or —OCH₃; and (b) instructions for treating the tauopathy or restless leg syndrome and/or an effective amount of a compound known for treating the tauopathy or restless leg syndrome.

In a further aspect, disclosed is a method of screening for positive allosteric modulators of the ability of a receptor-type tyrosine-protein phosphatase delta (PTPRD) to dephosphorylate a kinase, the method comprising: (a) contacting the PTPRD with a test compound in the presence of the phosphorylated kinase; and (b) measuring any orthophosphate release from the kinase; wherein the PTPRD comprises a phosphatase D1 domain having at least 80% amino acid identity with SEQ ID NO: 1; and wherein the kinase is GSKα or GSKβ comprising a polypeptide having at least 80% amino acid identity with SEQ ID NO: 2 or CDK5 comprising a polypeptide having at least 80% amino acid identity with SEQ ID NO: 4.

Still other objects and advantages of the present disclosure will become readily apparent by those skilled in the art from the following detailed description, which is shown and described by reference to preferred aspects, simply by way of illustration of the best mode. As will be realized, the disclosure is capable of other and different aspects, and its several details are capable of modifications in various respects, without departing from the disclosure. Accordingly, the description is to be regarded as illustrative in nature and not as restrictive.

### BRIEF DESCRIPTION OF THE DRAWINGS

The accompanying drawings, which are incorporated in and constitute a part of this specification and together with the description, serve to explain the principles of the disclosure.

FIG. 1 is a plot showing that PTPRD D1 phosphatase (SEQ ID NO: 1) liberates orthophosphate from pYGSK3β/α (triangles) and pY15 CDK5 (diamonds) to produce increasing malachite green/molybdate spectrophotometric signals at 605 nm over time as shown. Outlined symbols: vehicle added. Symbols without outline: 5×10⁻⁵M quercetin added. Phosphate liberation from END(pY)INASL control: squares.

FIG. 2 is an image showing high levels of PTPRD mRNA in Allen Brain Institute RNAseq datasets for most excitatory (R) and inhibitory (L) human cerebral cortical neuronal cell types. Many of these same cell types express GSK3β at moderate levels, several express CDK5 at moderate levels, a few express GSK3α at modest levels, several express the CDK5 binding partner CDK5R1 at moderate levels and a few express other CDK5 binding partners at modest-to-low levels. Scale: log 2 copies/million+1.

FIG. 3 is an image showing higher and more consistent levels of PTPRD mRNA in Allen Brain Institute RNAseq datasets across most excitatory (R) and inhibitory (L) human cerebral cortical neuronal cell types than any other receptor type protein tyrosine phosphatase, including the close PTPRD relatives PTPRS and PTPRF. Scale: log 2 copies/million+1.

FIG. 4 is a plot showing hydrolysis of the nonpeptide phosphatase substrate paranitrophenyl phosphate (pNPP; 1.8×10⁻⁵M) to p-nitrophenolate (405 nm absorption) by PTPRD D1 phosphatase (SEQ ID NO: 1): Competition by 4×10⁻⁴ M phosphoGSK, 4×10⁻⁴ M phosphoCDK5 and 5×10⁻⁵M of small molecule PTPRD phosphatase inhibitor, 7-BIA. See Uhl, G. R., et al., “Cocaine reward is reduced by decreased expression of receptor-type protein tyrosine phosphatase D (PTPRD) and by a novel PTPRD antagonist” Proc Natl Acad Sci USA, 2018. 115(45): p. 11597-11602.

FIG. 5 shows normalized rates of liberation of orthophosphate from pY15CDK5 wildtype and alanine substitution mutants by PTPRD D1 phosphatase. *nominal p value <0.05. P values were: 0.08, 0.004, 0.39, 0.09, 0.86, 0.009 and 0.012 (2 tailed t tests, Bonferoni corrected significance @ 0.007). Glutamic acid substitution mutants display trends (1, 4) and nominally-significant (6) reductions in activity. Lysine substitution mutants (2 and 7) display nominally-significant increases in activity. These differences fit with prior data from random sequence phosphopeptides. See N. G. Selner et al., “Diverse levels of sequence selectivity and catalytic efficiency of protein-tyrosine phosphatases.” Biochemistry 53, 397-412 (2014).

FIG. 6 includes plots and an image showing Western analyses and quantitation, revealing increased pY276 GSK3α (upper band, position (1)), pY216 GSK3β (lower band, position (2)) immunoreactivity in relation to β actin control (lowest band, position (3)) in proteins extracted from brains of wildtype (WT; L four lanes) vs heterozygous PTPRD knockout mice (Het; R four lanes). P values for two tailed t tests shown.

FIG. 7 shows normalized rates of orthophosphate release from pY15 CDK5 phosphopeptide by PTPRD phosphatase with addition of flavanols (quercetin, myricetin, morin, kaempferol, galangin, fisetin) or flavones (scutellarein, luteolin, chrysin, baicalien and apigenin) (10⁻⁴ M). Values normalized to control rates with vehicle added×100 (mean+/−SEM; * p=0.01, t test).

FIG. 8 shows normalized rates of orthophosphate release from pYGSK3 phosphopeptide by PTPRD phosphatase with addition of flavanols (quercetin, myricetin, morin, kaempferol, galangin, fisetin) or flavones (scutellarein, luteolin, chrysin, baicalien and apigenin) (10⁻⁴ M). Values normalized to control rates with vehicle added×100 (mean+/−SEM; * p<0.05; ** p<0.005, t test).

FIG. 9 is a plot showing dose-response relationship for quercetin stimulation of orthophosphate liberation from pYGSK3 (squares), pYCDK5 (diamonds) and control END(pY)INASL (circles) phosphopeptides by PTPRD phosphatase. Mean+/−SEM.

FIG. 10 shows a model of front (left) and back (right) views of PTPRD phosphatase (grey) with quercetin docked into the site at position (1) that provides the most favored binding score (−6.2 kcal/mol vs −4.5 and −4.8 kcal/mol for the sites where quercetin is depicted at position (3) (front and back views, respectively). Quercetin site at position (2) (center, front view) blocks PTPRD's phosphotyrosine binding/catalytic site.

FIG. 11 shows closer views of the quercetin binding to its highest affinity site (L side of L view in FIG. 10). Left: depiction of quercetin's six hydrogen bonds (dashes), one aromatic hydrogen bond and three pi-pi interactions with PTPRD's phosphatase. Right: view of quercetin's orientation in its most-energetically-favored binding pocket.

FIG. 12 shows an in silico model for PTPRD phosphatase with quercetin binding to its most energetically-favored site. This allows unimpeded CDK5 (bottom) and GSK3 (top) phosphopeptide recognition in “axial” (left) binding mode but impedes this recognition when phosphopeptide is in “equatorial” (right) binding modes. These positions represent the most favored of >1000 separate docking trials for quercetin and for the phosphopeptides, as noted above. “Axial” and “equatorial” are arbitrarily defined as noted above (methods). For orientation, note phosphotyrosine binding site catalytic cysteine.

### SEQUENCE LISTING

A list of relevant sequences is shown below in Table 1.

TABLE 1

| Sequence | Source |
|---|---|
| MHHHHHHASH PPIPILELAD HIERLKANDN LKFSQEYESI | phosphatase D1 domain of |
| DPGQQFTWEH SNLEVNKPKN RYANVIAYDH SRVLLSAIEG | receptor type protein |
| IPGSDYVNAN YIDGYRKQNA YIATQGSLPE TFGDFWRMIW | tyrosine phosphatase |
| EQRSATVVMM TKLEERSRVK CDQYWPSRGT | (PTPRD) |
| ETHGLVQVTL LDTVELATYC VRTFALYKNG SSEKREVRQF | |
| QFTAWPDHGV PEHPTPFLAF LRRVKTCNPP | |
| DAGPMVVHCS AGVGRTGCFI VIDAMLERIK HEKTVDIYGH | |
| VTLMRAQRNY MVQTEDQYIF IHDALLEAVT | |
| CGNTEVPARN L (SEQ ID NO: 1) | |
| QLVRGEPNVS-pY-ICSRYYRAPE (SEQ ID NO: 2) | GSK3β/α wildtype |
| | phosphopeptide |
| QLVRGEPNVSYICSRYYRAPE (SEQ ID NO: 3) | GSK3β/α wildtype |
| | dephosphopeptide |
| YEKLEKIGEGT-pY-GTVFKAKN (SEQ ID NO: 4) | CDK5 wildtype |
| | phosphopeptide |
| YEKLEKIGEGTYGTVFKAKN (SEQ ID NO: 5) | CDK5 wildtype |
| | dephosphopeptide |
| YEKLEKIGAGT-pY-GTVFKAKN (SEQ ID NO: 6) | CDK5 mutant 1 |
| | phosphopeptide. |
| YEKLEAIGEGT-pY-GTVFKAKN (SEQ ID NO: 7) | CDK5 mutant 2 |
| | phosphopeptide. |
| YEKLAKIGEGT-pY-GTVFKAKN (SEQ ID NO: 8) | CDK5 mutant 3 |
| | phosphopeptide |
| YEALEKIGEGT-pY-GTVFKAKN (SEQ ID NO: 9) | CDK5 mutant 4 |
| | phosphopeptide |
| YAKLEKIGEGT-pY-GTVFKAKN (SEQ ID NO: 10) | CDK5 mutant 5 |
| | phosphopeptide |
| YAKLAKIGAGT-pY-GTVFKAKN (SEQ ID NO: 11) | CDK5 mutant 6 |
| | phosphopeptide |
| YEALEAIGEGT-pY-GTVFKAKN (SEQ ID NO: 12) | CDK5 mutant 7 |
| | phosphopeptide |
| END-pY-INASL (SEQ ID NO: 13) | control phosphopeptide |

### DETAILED DESCRIPTION

The present invention can be understood more readily by reference to the following detailed description of the invention and the Examples included therein.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present compositions, methods, and kits may be understood more readily by reference to the following detailed description of preferred embodiments and the examples included therein.

While aspects of this disclosure can be described and claimed in a particular statutory class, such as the system statutory class, this is for convenience only and one of skill in the art will understand that each aspect of this disclosure can be described and claimed in any statutory class. Unless otherwise expressly stated, it is in no way intended that any method or aspect set forth herein be construed as requiring that its steps be performed in a specific order. Accordingly, where a method claim does not specifically state in the claims or description that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present application is not entitled to antedate such publication by virtue of prior invention. Further, stated publication dates may be different from actual publication dates, which can require independent confirmation.

### A. Definitions

Listed below are definitions of various terms used to describe this invention. These definitions apply to the terms as they are used throughout this specification, unless otherwise limited in specific instances, either individually or as part of a larger group.

As used in the specification and in the claims, the term “comprising” can include the aspects “consisting of” and “consisting essentially of.”

As used in the specification and claims, the singular forms “a,” “an,” and “the” include plural referents unless the context clearly dictates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the value designated some other value approximately or about the same. It is generally understood, as used herein, that it is the nominal value indicated ±10% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

“Optional” or “optionally” means that the subsequently described event or circumstance may or may not occur, and that the description includes instances where said event or circumstance occurs and instances where it does not.

The term “pharmaceutically acceptable salt,” as used herein, refers to an inorganic or organic salt of a disclosed compound or its derivative that is suitable for administration to a subject.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, and amides, salts of esters or amides, and N-oxides of a parent compound.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use.

As used herein, the term “by weight,” when used in conjunction with a component, unless specially stated to the contrary is based on the total weight of the formulation or composition in which the component is included. For example, if a particular element or component in a composition or article is said to have 8% by weight, it is understood that this percentage is in relation to a total compositional percentage of 100%.

A weight percent of a component, or weight %, or wt %, unless specifically stated to the contrary, is based on the total weight of the formulation or composition in which the component is included.

As used herein, the term “subject” can be a vertebrate, such as a mammal, a fish, a bird, a reptile, or an amphibian. Thus, the subject can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with an ailment, disease, or disorder. The term “patient” includes human and veterinary subjects.

As used herein, the terms “treatment” and “treating” refer to the medical management of a subject with the intent to cure, ameliorate, stabilize, or prevent an ailment, disease, pathological condition, disorder, or injury. This term includes active treatment, that is, treatment directed specifically toward the improvement of a disease, pathological condition, disorder, or injury, and also includes causal treatment, that is, treatment directed toward removal of the cause of the associated disease, pathological condition, disorder, or injury. In addition, this term includes palliative treatment, that is, treatment designed for the relief of symptoms rather than the curing of the disease, pathological condition, disorder, or injury; preventative treatment, that is, treatment directed to minimizing or partially or completely inhibiting the development of the associated disease, pathological condition, disorder, or injury; and supportive treatment, that is, treatment employed to supplement another specific therapy directed toward the improvement of the associated disease, pathological condition, disorder, or injury. In various aspects, the term covers any treatment of a subject, including a mammal (e.g., a human), and includes: (i) preventing the disorder or condition from occurring in a subject that can be predisposed to the disorder or condition but has not yet been diagnosed as having it; (ii) inhibiting the disorder or condition, i.e., arresting its development or exacerbation thereof; or (iii) relieving the disorder or condition, i.e., promoting healing of the disorder or condition. In one aspect, the subject is a mammal such as a primate, and, in a further aspect, the subject is a human.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by a compound as disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

“Tauopathy,” as used herein, refers to a heterogeneous group of neurodegenerative diseases characterized by abnormal metabolism of misfolded tau proteins leading to intracellular accumulation and formation of neurofibrillary tangles (NFT). Non-limiting examples of tauopathies include Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, and progressive supranuclear palsy.

“Restless leg syndrome” or “RLS,” as used herein, refers to a neurologic and sleep related movement disorder characterized by an irresistible urge to move in the legs that typically occurs or worsens at rest. RLS is usually accompanied by abnormal, uncomfortable sensations, known as paresthesias or dysesthesias, that are often likened to crawling, cramping, aching, burning, itching, or prickling deep within the affected areas.

“PTPRD,” as used herein, refers to a phosphatase enzyme known as receptor-type tyrosine-protein phosphatase delta, which is encoded by the PTPRD gene. The PTPRD enzyme contains an extracellular region, a single transmembrane segment, and two intracytoplasmic catalytic domains. The extracellular region of the enzyme comprises three Ig-like and eight fibronectin type III-like domains. The PTPRD enzyme is also known as HPTP, HPTPD, HPTPDELTA, PTPD, RPTDELTA, protein tyrosine phosphatase, receptor type D, protein tyrosine phosphatase receptor type D, and R-PTP-delta.

As used herein, “dosage form” means a pharmacologically active material in a medium, carrier, vehicle, or device suitable for administration to a subject.

A “compound known for treating” a stated disorder, as used herein, includes any compound known for treating the disorder, including on- and off-label uses approved by the U.S. Food and Drug Administration.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as a recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

References in the specification and concluding claims to parts by weight of a particular element or component in a composition or article, denotes the weight relationship between the element or component and any other elements or components in the composition or article for which a part by weight is expressed. Thus, in a composition or a selected portion of a composition containing 2 parts by weight of component X and 5 parts by weight component Y, X and Y are present at a weight ratio of 2:5, and are present in such ratio regardless of whether additional components are contained in the composition.

As used herein, the term “substantially,” in, for example, the context “substantially free of” refers to a composition having less than about 10% by weight, e.g., less than about 5%, less than about 1%, less than about 0.5%, less than about 0.1%, less than about 0.05%, or less than about 0.01% by weight of the stated material, based on the total weight of the composition.

It is further understood that the term “substantially,” when used in reference to a composition, refers to at least about 60% by weight, e.g., at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by weight, based on the total weight of the composition, of a specified feature, component, or a combination of the components. It is further understood that if the composition comprises more than one component, the two or more components can be present in any ratio predetermined by one of ordinary skill in the art.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

### B. Method of Treating a Tauopathy or Restless Leg Syndrome

In one aspect, disclosed is a method of treating a tauopathy or restless leg syndrome in a subject, comprising administering to the subject an effective amount of a compound represented by Formula (I):

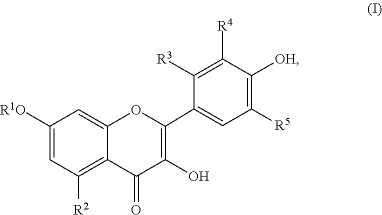

or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

According to one aspect, the compound administered to the subject is one of the following compounds or a pharmaceutically acceptable salt thereof:

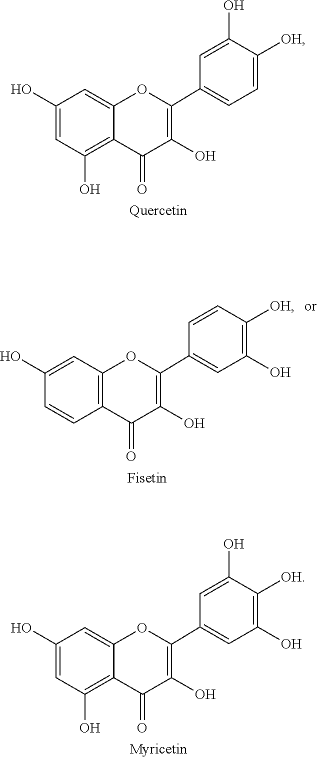 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

In a further aspect, the compound is Quercetin or a pharmaceutically acceptable salt thereof.

Compounds of Formula (I) can be administered to the subject as a pharmaceutically-acceptable salt. Non-limiting examples of pharmaceutically-acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. Other non-limiting examples include, but are not limited, to sulfate, citrate, acetate, oxalate, chloride, bromide, iodide, nitrate, bisulfate, phosphate, acid phosphate, phosphonic acid, isonicotinate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Still other salts include, but are not limited to, salts with inorganic bases including alkali metal salts such as sodium salts, and potassium salts; alkaline earth metal salts such as calcium salts, and magnesium salts; aluminum salts; and ammonium salts. Other salts with organic bases include salts with diethylamine, diethanolamine, meglumine, and N,N′-dibenzylethylenediamine. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

Pharmaceutically-acceptable salts of compounds of Formula (I) can be salts formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts. Similarly, acid addition salts, such as mineral acids, organic carboxylic and organic sulfonic acids, e.g., hydrochloric acid, methanesulfonic acid, maleic acid, are also contemplated. Neutral forms of the compounds can be regenerated by contacting the salt with a base or acid and isolating the parent compound in a conventional manner.

In one aspect, a compound of Formula (I) or a pharmaceutically acceptable salt thereof can be administered to a subject having a tauopathy or restless leg syndrome. According to one aspect, the tauopathy is Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, or progressive supranuclear palsy. In one aspect, a compound of Formula (I) or a pharmaceutically acceptable salt thereof can be administered to a subject having Alzheimer's disease. In a further aspect, a compound of Formula (I) or a pharmaceutically acceptable salt thereof can be administered to a subject having restless leg syndrome.

Compounds of Formula (I) and pharmaceutically acceptable salts thereof can be administered to the subject via a variety of routes. Non-limiting examples include oral administration (e.g., as a tablet, capsule, lozenge, or troche) or intravenous administration of the compound or pharmaceutically acceptable salt thereof together with a pharmaceutically-acceptable carrier.

The effective amount or dosage of the composition or an ingredient thereof can vary within wide limits. Such a dosage is adjusted to the individual requirements in each particular case including the specific composition(s) being administered and the condition being treated, as well as the subject being treated. In general, single dose compositions can contain such amounts or submultiples thereof of the composition to make up the daily dose. The dosage can be adjusted by the individual physician in the event of any contraindications. Dosage can vary, and can be administered in one or more dose administrations daily, for one or several days. In some aspects, the effective amount is a therapeutically-effective amount. In a further aspect, the effective amount is a prophylactically-effective amount.

In one aspect, the subject to be treated is a mammal. In a further aspect, the subject is a human. In a still further aspect, the subject has been diagnosed with a need for treatment of the tauopathy or restless leg syndrome prior to the administering step. In a further aspect, the treatment method comprises the step of identifying a subject in need of treatment of the tauopathy or restless leg syndrome prior to the administering step.

1. Pharmaceutically-Acceptable Carriers and Dosage Forms

In various aspects, compounds of Formula (I) or a pharmaceutically acceptable salt thereof can be administered to the subject as a composition or formulation comprising a pharmaceutically-acceptable carrier. Non-limiting examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

Pharmaceutically-acceptable carries can also comprise adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms can be made by forming microencapsule matrices of the compound in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations can also be prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose.

In some aspects, the pharmaceutically-acceptable carrier can include an excipient. Suitable excipients include, without limitation, saccharides, for example, glucose, lactose, or sucrose, mannitol, or sorbitol, cellulose derivatives, and/or calcium phosphate, for example, tricalcium phosphate or acidic calcium phosphate.

In further aspects, the pharmaceutically-acceptable carrier can include a binder. Suitable binders include, without limitation, tare compounds such as starch paste, for example, corn, wheat, rice, and potato starch, gelatin, tragacanth, methylcellulose, hydroxypropyl methylcellulose, carboxymethylcellulose, and/or polyvinylpyrrolidone. In still further aspects, there can be a disintegrating agent, such as the aforementioned starches and carboxymethyl starch, crosslinked polyvinylpyrrolidone, agar, or alginic acid or a salt thereof, such as sodium alginate.

In some aspects, the pharmaceutically-acceptable carrier can include an additive. Examples of additives include, but are not limited to, diluents, buffers, binders, surface-active agents, lubricants, humectants, pH adjusting agents, preservatives (including anti-oxidants), emulsifiers, occlusive agents, opacifiers, antioxidants, colorants, flavoring agents, gelling agents, thickening agents, stabilizers, and surfactants, among others. Thus, in various further aspects, the additive is vitamin E, gum acacia, citric acid, stevia extract powder, Luo Han Gou, Monoammonium Glycyrhizinate, Ammonium Glycyrrhizinate, honey, or combinations thereof. In a still further aspect, the additive is a flavoring agent, a binder, a disintegrant, a bulking agent, or silica. In a further aspect, the additive can include flowability-control agents and lubricants, such as silicon dioxide, talc, stearic acid and salts thereof, such as magnesium stearate or calcium stearate, and/or propylene glycol.

In various aspects, when compounds of Formula (I) or a pharmaceutically acceptable salt thereof are formulated for oral use, such as for example, a tablet, pill, or capsule, the composition can include a coating layer that is resistant to gastric acid. Such a layer, in various aspects, can include a concentrated solution of saccharides that can comprise gum arabic, talc, polyvinylpyrrolidone, polyethylene glycol, and/or titanium dioxide, and suitable organic solvents or salts thereof.

Dosage forms can comprise a compound of Formula (I) or a pharmaceutically acceptable salt thereof, together in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed composition or a product of a disclosed method of making, suspended in sterile saline solution for injection together with a preservative.

2. Combination Therapies

Also disclosed herein is a combination therapy comprising administering to a subject having a tauopathy or restless leg syndrome a compound of Formula (I) or a pharmaceutically acceptable salt thereof, together with a compound known for treating the tauopathy or restless leg syndrome. In one aspect, when the subject has a tauopathy, a compound of Formula (I) or a pharmaceutically acceptable salt thereof can be co-administered with a compound known for treating the tauopathy. For example, many amyloid-β-targeting therapies have been evaluated for possible efficacy against tauopathies including Alzheimer's disease. Without wishing to be bound by theory, it is believed that the compounds of Formula (I) and pharmaceutically acceptable salts thereof may increase the effectiveness of known amyloid-β-targeting therapies among other therapies targeting tauopathies such as Alzheimer's disease.

In one aspect, the treatment method can comprise administering to the subject having a tauopathy an effective amount of a compound of Formula (I), or a pharmaceutically acceptable salt thereof, together with an amyloid-β-targeting therapy. Non-limiting examples of amyloid-β-targeting therapies include drugs that bind to various forms of amyloid-producing peptides or amyloid, including but not limited to MEDI1814 (AN1814), LY2599666, PF-05236812 (AAB-003), LY3002813 (Donanemab), BAN2401, Ponezumab (PF-04360365), GSK933776, Solanezumab, Aducanumab, Crenezumab, Gantenerumab and Bapineuzumab. Other examples include drugs that induce host antibodies against amyloid producing peptides or amyloid, including but not limited to LuAF20513, ABvac 40, UB 311, ACI-24, Vanutide cridificar, AN-1792, Affitope AD02, CAD106 (Amilomotide), and CAD106. Still further examples include drugs that inhibit or modulate gamma secretase, including but not limited to PF-06648671, Begacestat (GSI-953), Avagacestat (BMS708163), EVP-0962, NIC5-15, and Semagacestat (LY450139). Further examples include drugs that inhibit beta secretase, including but not limited to Lanabecestat (AZD3293 or LY3314814), BI 1181181 (VTP 37948), RG7129, LY2886721, LY3202626, Elenbecestat, CNP520 (Umibecestat), Verubecestat (MK-893), Atabecestat (JNJ-54861911), and Lanabecestat (AZD3293 or LY3314814). Additionally, non-limiting examples of suitable tau-targeting therapies that can be used in combination with the compounds of Formula (I) include without limitation Gosuranemab (BIIB092) and Semorinemab (RG 6100).

In a further aspect, the compounds of Formula (I) can be used in combination with therapies targeting other mechanisms in tauopathies and Alzheimer's disease, including but not limited to PQ912, CT1812, Acitretin, Thalidomide, Bexarotene, Clioquinol, Epigallacatechin gallate, Scyllo-inositol Etazolate, Immunoglobin+albumin, Sodium oligomannurarate (GV-971), Tarenflurbill, Intravenous immunoglobulin, Tramiprosate (homotaurine), Alicapistat (ABT-957), ABT-354, PF-05212377, SB-659032 (Rilapladib), AD-35, filgrastim, DHP1401, edonerpic maleate (T-817MA), carvedilol, AR1001, TC-5619, TPI 287, Intepirdine (SB-742457 or RVT-101), ORY-2001 (Vafidemstat), Benfotiamine (thiamine), Piromelatine (Neu-P11), Memnatine, Octohydroaminoacridine, Pepinema, Azeliragon (TTP488), Dapagliflozin, GSK239512, Thiethylperazine, ASP0777, Montelukast, PF-04447943, HF 0220, PTI-125, Perindopril, Telmisartan, Posiphen, S-equol, leuprolide, 3APS, and LY3372993.

According to one aspect, when the subject has restless leg syndrome, a compound of Formula (I) or a pharmaceutically acceptable salt thereof can be co-administered to the subject with a compound known for treating restless leg syndrome. In one aspect, the compound known for treating restless leg syndrome can be a drug known to affect dopamine levels. In some aspects, the compound can act via direct dopamine replacement, e.g., through a drug known as levodopa. In a further aspect, the compound known for treating restless leg syndrome can be a dopamine agonist, e.g., pramipexole, ropinirole, rotigotine, or a combination thereof. Other suitable compounds for treating restless leg syndrome include without limitation bromocryptine, pergolide, cabergoline, or a combination thereof. In a further aspect, the compound known for treating restless leg syndrome can be gabapentin. In a still further aspect, the compound known for treating restless leg syndrome can be a benzodiazepine such as clonazepan or diazepam. In a still further aspect, the compound known for treating restless leg syndrome can be an opiate agonist such as codeine or tramadol.

### C. Manufacture of a Medicament

Also disclosed is the use of a compound of Formula (I) or a pharmaceutically acceptable salt thereof in the manufacture of a medicament for the treatment of a tauopathy or restless leg syndrome:

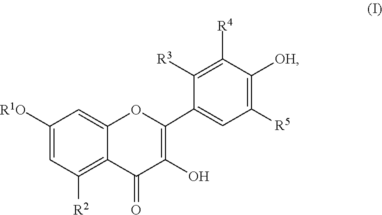

wherein R¹ is hydrogen or —CH₃; R², R³, and R⁵ are each independently hydrogen or —OH; and R⁴ is hydrogen, —OH, or —OCH₃.

According to one aspect, disclosed is the use of one of the following compounds or a pharmaceutically acceptable salt thereof in the manufacture of a medicament for the treatment of a tauopathy or restless leg syndrome:

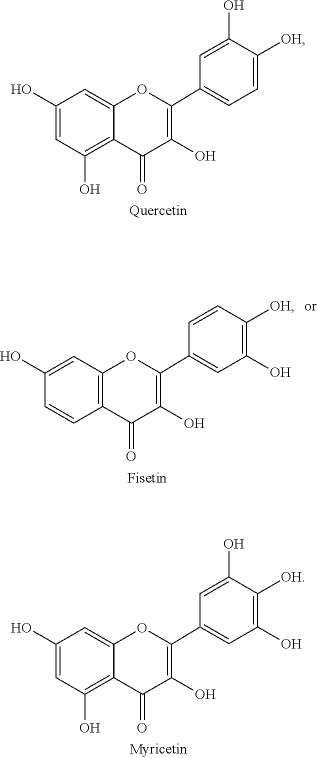 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

Also disclosed herein is the use of a compound of Formula (I) or a pharmaceutically acceptable salt thereof, together with a compound or agent known for treating a tauopathy or restless leg syndrome, in the manufacture of a medicament. In one aspect, for example, when the subject has a tauopathy, disclosed is the use of a compound of Formula (I) or a pharmaceutically acceptable salt thereof along with a compound known for treating the tauopathy. For example, many amyloid-β-targeting therapies have been evaluated for possible efficacy against tauopathies including Alzheimer's disease. Without wishing to be bound by theory, it is believed that the compounds of Formula (I) and pharmaceutically acceptable salts thereof may increase the effectiveness of known amyloid-β-targeting therapies among other therapies targeting tauopathies such as Alzheimer's disease.

In one aspect, the manufacture of the medicament can comprise co-formulating or co-packaging a compound of Formula (I), or a pharmaceutically acceptable salt thereof, together with a therapy targeting amyloid-β, tau, or other tauopathy or Alzheimer's disease mechanism. Non-limiting examples include drugs that bind to various forms of amyloid-producing peptides or amyloid, including but not limited to MEDI1814 (AN1814), LY2599666, PF-05236812 (AAB-003), LY3002813 (Donanemab), BAN2401, Ponezumab (PF-04360365), GSK933776, Solanezumab, Aducanumab, Crenezumab, Gantenerumab and Bapineuzumab. Other examples include drugs that induce host antibodies against amyloid producing peptides or amyloid, including but not limited to LuAF20513, ABvac 40, UB 311, ACI-24, Vanutide cridificar, AN-1792, Affitope AD02, CAD106 (Amilomotide), and CAD106. Still further examples include drugs that inhibit or modulate gamma secretase, including but not limited to PF-06648671, Begacestat (GSI-953), Avagacestat (BMS708163), EVP-0962, NIC5-15, and Semagacestat (LY450139). Further examples include drugs that inhibit beta secretase, including but not limited to Lanabecestat (AZD3293 or LY3314814), BI 1181181 (VTP 37948), RG7129, LY2886721, LY3202626, Elenbecestat, CNP520 (Umibecestat), Verubecestat (MK-893), Atabecestat (JNJ-54861911), and Lanabecestat (AZD3293 or LY3314814). Additionally, non-limiting examples of suitable tau-targeting therapies that can be used in combination with the compounds of Formula (I) include without limitation Gosuranemab (BIIB092) and Semorinemab (RG 6100).

In a further aspect, the compounds of Formula (I) can be used co-formulated or co-packaged with therapies targeting other mechanisms in tauopathies and Alzheimer's disease, including but not limited to PQ912, CT1812, Acitretin, Thalidomide, Bexarotene, Clioquinol, Epigallacatechin gallate, Scyllo-inositol Etazolate, Immunoglobin+albumin, Sodium oligomannurarate (GV-971), Tarenflurbill, Intravenous immunoglobulin, Tramiprosate (homotaurine), Alicapistat (ABT-957), ABT-354, PF-05212377, SB-659032 (Rilapladib), AD-35, filgrastim, DHP1401, edonerpic maleate (T-817MA), carvedilol, AR1001, TC-5619, TPI 287, Intepirdine (SB-742457 or RVT-101), ORY-2001 (Vafidemstat), Benfotiamine (thiamine), Piromelatine (Neu-P11), Memnatine, Octohydroaminoacridine, Pepinema, Azeliragon (TTP488); Dapagliflozin, GSK239512, Thiethylperazine, ASP0777, Montelukast, PF-04447943, HF 0220, PTI-125, Perindopril, Telmisartan, Posiphen, S-equol, leuprolide, 3APS, and LY3372993.

According to one aspect, a compound of Formula (I) or a pharmaceutically acceptable salt thereof can be co-formulated or co-packaged with a compound known for treating restless leg syndrome. In one aspect, the compound known for treating restless leg syndrome can be a drug known to affect dopamine levels. In some aspects, the compound can act via direct dopamine replacement, e.g., through a drug known as levodopa. In a further aspect, the compound known for treating restless leg syndrome can be a dopamine agonist, e.g., pramipexole, ropinirole, rotigotine, or a combination thereof. Other suitable compounds for treating restless leg syndrome include without limitation bromocryptine, pergolide, cabergoline, or a combination thereof. In a further aspect, the compound known for treating restless leg syndrome can be gabapentin. In a still further aspect, the compound known for treating restless leg syndrome can be a benzodiazepine such as clonazepan or diazepam. In a still further aspect, the compound known for treating restless leg syndrome can be an opiate agonist such as codeine or tramadol.

In various aspects, the method for the manufacture of a medicament comprises combining a therapeutically effective amount of a disclosed compound of Formula (I), or a pharmaceutically acceptable salt thereof, with a pharmaceutically acceptable carrier or diluent and/or with a compound known for treating the tauopathy or restless leg syndrome. In a further aspect, disclosed is a method for the manufacture of a medicament for treating a tauopathy or restless leg syndrome, the method comprising combining a therapeutically effective amount of a disclosed compound of Formula (I) or a pharmaceutically acceptable salt thereof with a therapeutically effective amount of a compound known for treating the tauopathy or restless leg syndrome, together with a pharmaceutically acceptable carrier or diluent.

### D. Kits

In a further aspect, disclosed is a kit comprising (a) a compound represented by Formula (I) in an amount effective for treating a tauopathy or restless leg syndrome in a subject,

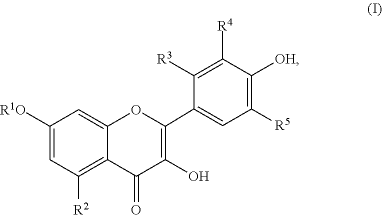

or a pharmaceutically acceptable salt thereof; wherein R¹ is hydrogen or —CH₃; R², R³, and R⁵ are each independently hydrogen or —OH; and R⁴ is hydrogen, —OH, or —OCH₃; and (b) instructions for treating the tauopathy or restless leg syndrome and/or an effective amount of a compound known for treating the tauopathy or restless leg syndrome.

According to one aspect, the kit comprises one of the following compounds or a pharmaceutically acceptable salt thereof:

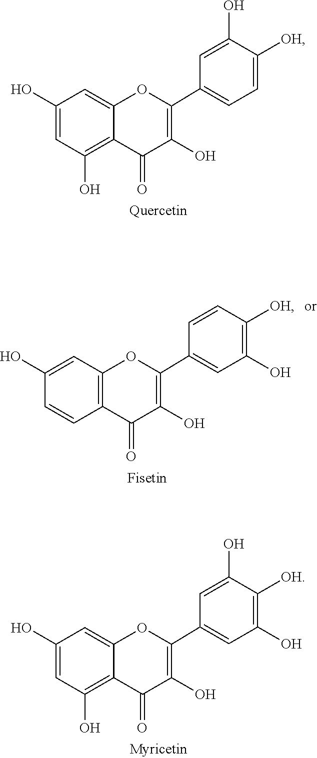 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

In a further aspect, the kit comprises Quercetin or a pharmaceutically acceptable salt thereof.

In some aspects, when the kit comprises instructions, the instructions can be suitable for a tauopathy such as Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, or progressive supranuclear palsy. In a further aspect, when the kit comprises instructions, the instructions can be suitable for restless leg syndrome. The instructions can be appropriate for a variety of subjects, e.g., a mammal or a human.

In one aspect, the kit can comprise a compound of Formula (I), or a pharmaceutically acceptable salt thereof, together with a therapy targeting amyloid-β, tau, or other tauopathy or Alzheimer's disease mechanism. Non-limiting examples include drugs that bind to various forms of amyloid-producing peptides or amyloid, including but not limited to MEDI1814 (AN1814), LY2599666, PF-05236812 (AAB-003), LY3002813 (Donanemab), BAN2401, Ponezumab (PF-04360365), GSK933776, Solanezumab, Aducanumab, Crenezumab, Gantenerumab and Bapineuzumab. Other examples include drugs that induce host antibodies against amyloid producing peptides or amyloid, including but not limited to LuAF20513, ABvac 40, UB 311, ACI-24, Vanutide cridificar, AN-1792, Affitope AD02, CAD106 (Amilomotide), and CAD106. Still further examples include drugs that inhibit or modulate gamma secretase, including but not limited to PF-06648671, Begacestat (GSI-953), Avagacestat (BMS708163), EVP-0962, NIC5-15, and Semagacestat (LY450139). Further examples include drugs that inhibit beta secretase, including but not limited to Lanabecestat (AZD3293 or LY3314814), BI 1181181 (VTP 37948), RG7129, LY2886721, LY3202626, Elenbecestat, CNP520 (Umibecestat), Verubecestat (MK-893), Atabecestat (JNJ-54861911), and Lanabecestat (AZD3293 or LY3314814). Additionally, non-limiting examples of suitable tau-targeting therapies that can be used in combination with the compounds of Formula (I) include without limitation Gosuranemab (BIIB092) and Semorinemab (RG 6100).

In a further aspect, the kit can comprise a compound of Formula (I), or a pharmaceutically acceptable salt thereof, together with a therapy targeting other mechanisms in tauopathies and Alzheimer's disease, including but not limited to PQ912, CT1812, Acitretin, Thalidomide, Bexarotene, Clioquinol, Epigallacatechin gallate, Scyllo-inositol Etazolate, Immunoglobin+albumin, Sodium oligomannurarate (GV-971), Tarenflurbill, Intravenous immunoglobulin, Tramiprosate (homotaurine), Alicapistat (ABT-957), ABT-354, PF-05212377, SB-659032 (Rilapladib), AD-35, filgrastim, DHP1401, edonerpic maleate (T-817MA), carvedilol, AR1001, TC-5619, TPI 287, Intepirdine (SB-742457 or RVT-101), ORY-2001 (Vafidemstat), Benfotiamine (thiamine), Piromelatine (Neu-P11), Memnatine, Octohydroaminoacridine, Pepinema, Azeliragon (TTP488); Dapagliflozin, GSK239512, Thiethylperazine, ASP0777, Montelukast, PF-04447943, HF 0220, PTI-125, Perindopril, Telmisartan, Posiphen, S-equol, leuprolide, 3APS, and LY3372993.

According to one aspect, the kit can comprise a compound of Formula (I) or a pharmaceutically acceptable salt thereof together with a compound known for treating restless leg syndrome. In one aspect, the compound known for treating restless leg syndrome can be a drug known to affect dopamine levels. In some aspects, the compound can act via direct dopamine replacement, e.g., through a drug known as levodopa. In a further aspect, the compound known for treating restless leg syndrome can be a dopamine agonist, e.g., pramipexole, ropinirole, rotigotine, or a combination thereof. Other suitable compounds for treating restless leg syndrome include without limitation bromocryptine, pergolide, cabergoline, or a combination thereof. In a further aspect, the compound known for treating restless leg syndrome can be gabapentin. In a still further aspect, the compound known for treating restless leg syndrome can be a benzodiazepine such as clonazepan or diazepam. In a still further aspect, the compound known for treating restless leg syndrome can be an opiate agonist such as codeine or tramadol.

In one aspect, the compound of Formula (I) or a pharmaceutically acceptable salt thereof and/or the compound known for treating the tauopathy or restless leg syndrome can be present in the kit in a therapeutically effective amount. In a further aspect, the compound of Formula (I) or a pharmaceutically acceptable salt thereof and/or the compound known for treating the tauopathy or restless leg syndrome can be present in the kit in a prophylactically effective amount.

In various aspects, the compound of Formula (I) or a pharmaceutically-acceptable salt thereof, the instructions for the use thereof (when present) and/or a combination therapy including a compound known for treating the target condition can be co-packaged and/or co-formulated. In a still further aspect, the compound or pharmaceutically-acceptable salt thereof, the instructions (when present), and/or the compound known for treating the target condition are not co-packaged.

The kits can also comprise compounds and/or products co-packaged, co-formulated, and/or co-delivered with other components. For example, a drug manufacturer, a drug reseller, a physician, a compounding shop, or a pharmacist can provide a kit comprising a disclosed compound and/or product and another component for delivery to a patient.

It is understood that the disclosed kits can be prepared from the disclosed compounds and pharmaceutical formulations. It is also understood that the disclosed kits can be employed in connection with the disclosed methods of using the compounds and pharmaceutical formulations.

### E. Method of Enhancing PTPRD's Ability to Dephosphorylate Kinase

In a further aspect, disclosed is a method of enhancing the ability of receptor-type tyrosine-protein phosphatase delta (PTPRD) to dephosphorylate a kinase, the method comprising contacting PTPRD with an effective amount of a compound represented by Formula (I):

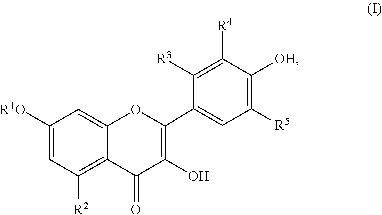

or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

In one aspect, the compound is:

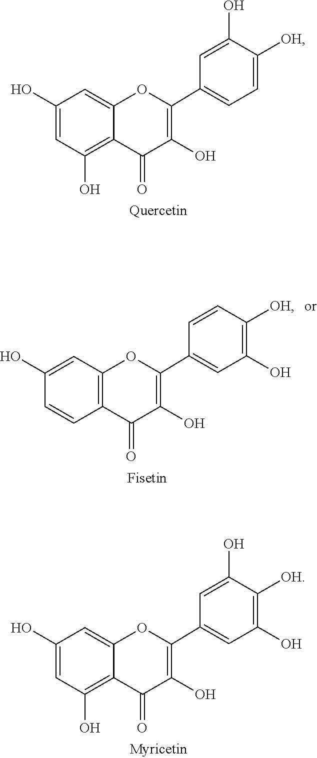 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

In a further aspect, the compound is Quercetin.

In one aspect, the PTPRD comprises a phosphatase D1 domain having at least 80% amino acid identity with SEQ ID NO: 1. In a further aspect, the PTPRD comprises a phosphatase D1 domain having at least 85% amino acid identity with SEQ ID NO: 1. In a further aspect, the PTPRD comprises a phosphatase D1 domain having at least 90% amino acid identity with SEQ ID NO: 1. In a further aspect, the PTPRD comprises a phosphatase D1 domain having at least 95% amino acid identity with SEQ ID NO: 1. In a still further aspect, the phosphatase D1 domain is SEQ ID NO: 1.

In some aspects, the kinase is glycogen synthase kinase GSK3β, glycogen synthase kinase GSK3α, cyclin dependent kinase-5 CDK5, or a combination thereof. According to one aspect, the kinase is GSKα or GSKβ comprising a polypeptide having at least 80% amino acid identity with SEQ ID NO: 2 or CDK5 comprising a polypeptide having at least 80% amino acid identity with SEQ ID NO: 4. In a further aspect, the kinase is GSKα or GSKβ comprising a polypeptide having at least 85% amino acid identity with SEQ ID NO: 2 or CDK5 comprising a polypeptide having at least 85% amino acid identity with SEQ ID NO: 4. In a further aspect, the kinase is GSKα or GSKβ comprising a polypeptide having at least 90% amino acid identity with SEQ ID NO: 2 or CDK5 comprising a polypeptide having at least 90% amino acid identity with SEQ ID NO: 4. In a further aspect, the kinase is GSKα or GSKβ comprising a polypeptide having at least 95% amino acid identity with SEQ ID NO: 2 or CDK5 comprising a polypeptide having at least 95% amino acid identity with SEQ ID NO: 4. In a still further aspect, the kinase is GSKα or GSKβ comprising a polypeptide that is SEQ ID NO: 2 or CDK5 comprising a polypeptide that is SEQ ID NO: 4.

### F. Screening Method

Also disclosed herein is a method of screening for positive allosteric modulators of the ability of a receptor-type tyrosine-protein phosphatase delta (PTPRD) to dephosphorylate a kinase. The method can comprise (a) contacting the PTPRD with a test compound in the presence of the phosphorylated kinase; and (b) measuring any orthophosphate release from the kinase; wherein the PTPRD comprises a phosphatase D1 domain having at least 80% amino acid identity with SEQ ID NO: 1; and wherein the kinase is GSKα or GSKβ comprising a polypeptide having at least 80% amino acid identity with SEQ ID NO: 2 or CDK5 comprising a polypeptide having at least 80% amino acid identity with SEQ ID NO: 4.

In one aspect, the PTPRD used in the screening method comprises a phosphatase D1 domain having at least 80% amino acid identity with SEQ ID NO: 1. In a further aspect, the PTPRD comprises a phosphatase D1 domain having at least 85% amino acid identity with SEQ ID NO: 1. In a further aspect, the PTPRD comprises a phosphatase D1 domain having at least 90% amino acid identity with SEQ ID NO: 1. In a further aspect, the PTPRD comprises a phosphatase D1 domain having at least 95% amino acid identity with SEQ ID NO: 1. In a still further aspect, the phosphatase D1 domain is SEQ ID NO: 1.

In some aspects, the kinase used in the screening method is glycogen synthase kinase GSK3β, glycogen synthase kinase GSK3α, cyclin dependent kinase-5 CDK5, or a combination thereof. According to one aspect, the kinase is GSKα or GSKβ comprising a polypeptide having at least 80% amino acid identity with SEQ ID NO: 2 or CDK5 comprising a polypeptide having at least 80% amino acid identity with SEQ ID NO: 4. In a further aspect, the kinase is GSKα or GSKβ comprising a polypeptide having at least 85% amino acid identity with SEQ ID NO: 2 or CDK5 comprising a polypeptide having at least 85% amino acid identity with SEQ ID NO: 4. In a further aspect, the kinase is GSKα or GSKβ comprising a polypeptide having at least 90% amino acid identity with SEQ ID NO: 2 or CDK5 comprising a polypeptide having at least 90% amino acid identity with SEQ ID NO: 4. In a further aspect, the kinase is GSKα or GSKβ comprising a polypeptide having at least 95% amino acid identity with SEQ ID NO: 2 or CDK5 comprising a polypeptide having at least 95% amino acid identity with SEQ ID NO: 4. In a still further aspect, the kinase is GSKα or GSKβ comprising a polypeptide that is SEQ ID NO: 2 or CDK5 comprising a polypeptide that is SEQ ID NO: 4.

According to one aspect, the test compound screened for activity is a flavanoid. In a further aspect, the test compound is a flavanol.

### G. Examples

The following examples are put forth so as to provide those of ordinary skill in the art with a complete disclosure and description of how the methods and products claimed herein are made and evaluated, and are intended to be purely exemplary and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. The Examples are provided herein to illustrate the invention, and should not be construed as limiting the invention in any way.

1. Phosphatases, Phosphopeptides, and Flavanoids

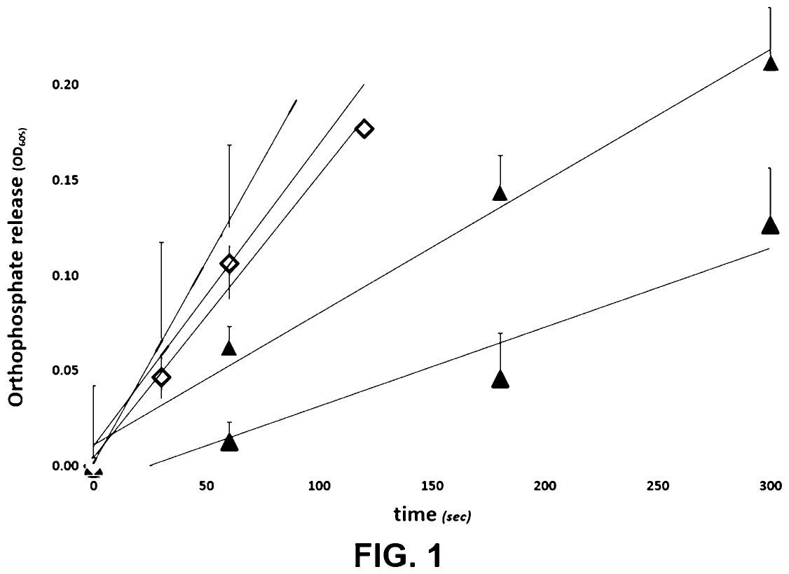

The following examples utilized phosphatase protein produced from a His-tagged D1 phosphatase domain synthesized expression vector optimized for E. Coli codon use (SEQ ID NO: 1). This protein was purified to >95% purity and was active in hydrolyzing pNPP substrate (FIG. 1). Human wildtype and mutant phospho- and dephospho-GSK3β/GSK3α and CDK5 peptides were synthesized (Pierce/ThermoFisher, SEQ ID NOs: 2-12) and control END(pY)INASL peptide (Promega, SEQ ID NO: 13) was purchased. Flavonoids were purchased from Thermo-Fisher, Aldrich, Cayman Chemical and AK Scientific. Purities were >95%.

2. Phosphatase Assays

pNPP dephosphorylation to p-nitrophenolate: Triplicate assays were used for human PTPRD phosphatase, pNPP substrate and spectrophotometric 405 nm detection of the dephosphorylation product of these enzyme's activities using a Spectromax plate reader. Controls used 5×10⁻⁵M 7-BIA with 18 min incubations as described. See G. R. Uhl et al., “Cocaine reward is reduced by decreased expression of receptor-type protein tyrosine phosphatase D (PTPRD) and by a novel PTPRD antagonist.” Proc Natl Acad Sci USA 115, 11597-11602 (2018). For assays determining the effect of flavonoids on pNPP hydrolysis, a 96 well half-area plate was prepared by first filling each well to be tested with 18 μL of 50 μM pNPP and 25 μL of running buffer (43.4 μM HEPES (pH 7.4), 2.2 μM dithiothreitol, 0.44% acetylated bovine serum albumin, 22.2 μM NaCl, 4.4 μM EDTA), and 2 μL of DMSO containing the desired concentration of flavonoid and/or peptide. PTPRD1 was diluted 1:50 in a dilution buffer (22.9 μM pH 7.4 HEPES, 1% acetylated bovine serum albumin, 4.6 μM dithiothreitol). At the zero time point, 5 uL of this solution was added to each well, and the optical density was measured in 36 second intervals at 405 nm. Results were plotted and the slopes of the linear region were fit. All experiments were performed three times with three wells dedicated to each experimental condition in each experiment.

Orthophosphate release assays (Promega V2471) used Malachite green and molybdate with spectrophotometric detection of liberated free orthophosphate from test phosphopeptides compared to control and mutant peptides with assessments for the times indicated. Reactions were carried out in a half-area 96-well plate, with three wells dedicated for each time point. To each experimental well, we added a mixture of 18 μL of ultrapure water, 25 μL of running buffer, 1 μL of a 10 mM DMSO solution of the desired peptide, and 1 μL of DMSO containing a desired concentration of flavonoid, or 1 μL DMSO for control experiments. The supplied molybdate dye mixture (50 μL) was added at t=0, followed by 5 μL of a 1:100 dilution of enzyme in the aforementioned dilution buffer. Other wells were initiated via the addition of 5 μL of the diluted enzyme mixture @ t=0 and terminated at the desired timepoints by addition of 50 μL of the dye solution. Wells were read @ 605 nm.

3. Ptprd Knockout Mice

PTPRD heterozygous knockout and wildtype littermates were obtained from heterozygote x heterozygote crosses, genotyped using gel analyses of PCR products of DNA extracted from ear punches and maintained in AALAC-certified facility with free access to food and water as described. See J. Drgonova et al., Mouse model for PTPRD associations with WED/RLS and addiction: reduced expression alters locomotion, sleep behaviors and cocaine-conditioned place preference. Mol Med, (2015); G. R. Uhl et al., Cocaine reward is reduced by decreased expression of receptor-type protein tyrosine phosphatase D (PTPRD) and by a novel PTPRD antagonist. Proc Natl Acad Sci USA 115, 11597-11602 (2018). 8-12 week old mice were euthanized by fast cervical dislocation and decapitation, brains removed by rapid dissection, rinsed with ice cold PBS, trimmed to remove olfactory bulb and cerebellum, frozen by dry ice/ethanol bath and stored at −80° C.

4. Western Analyses

Protein were extracted from frozen tele/di/mesencephalic brain samples using a hand held sonicator (Branson) in 20 ml/g wet weight T-PER (Thermo Scientific) with 1:1000 complete mini protease inhibitor cocktail (Roche) and 1 tab/10 ml phosphatase inhibitor cocktail set II (Calbiochem). Proteins in supernatant from 10,000×g/30 min/4° C. centrifugation were separated by electrophoresis under reducing conditions using precast gels (PCG2012 TruPAGE, Sigma) and transferred to nitrocellulose membranes (88018 Thermo Scientific). Membranes were preincubated for 30 min in 5% nonfat milk in Tris-buffered saline/Tween (TBST: 0.1M Tris, 0.15 M NaCl, and 0.1% Tween 20), incubated with primary antibodies (rabbit anti pY15 CDK5 (Sigma) or rabbit anti pY279 GSK3α/β (Millipore) overnight at 4° C. in 5% milk in TBST buffer, washed 3×/10 min in TBST, incubated with secondary antibody (925-32211 LiCOR) for 1 h at 22° C., washed 3× in TBST, imaged and quantified (LI-COR Odyssey; LI-COR Biosciences).

5. Peptide Docking

Dephospho CDK5 and GSK3 peptides were docked to PTPRD (PDB ID 2NV5 using FlexPepDock and the ab-initio protocol that folds the peptide as it docks. A library of 3, 5, and 9-amino acid backbone fragments was generated, a linear peptide superimposed near the active site and the complex prepacked. 100,000 models were generated as described. See B. Raveh, N. London, L. Zimmerman, O. Schueler-Furman, Rosetta FlexPepDock ab-initio: simultaneous folding, docking and refinement of peptides onto their receptors. PloS one 6, e18934 (2011). Docked models were discarded if the tyrosine was not oriented for catalysis as in the structure of PTPRC bound to phosphopeptide substrate (PDB ID 1YGR). Models were thus discarded if the hydroxyl oxygen was more than 6 Å away from the catalytic cysteine or if the gamma carbon was more than 2 Å from the gamma carbon of the phosphotyrosine bound to PTPRC (after alignment to PTPRD). 100,000 models were generated for each substrate. 2,060 CDK5 and 712 GSK3 models satisfied the reweighted_sc scoring term, as recommended for ab-initio docking. Each of the top 10 models oriented peptide backbones in modes that we arbitrarily define as “axial” (eg the axis of the plane that passes through α7 helix and the catalytic cysteine) or “equatorial” modes (orthogonal to the axial mode). We added phosphates to the tyrosines in the top scoring pose for each peptide orientation. These phosphopeptides were then docked and generated 1,100 models using the FlexPepDock refinement protocol. See B. Raveh, N. London, O. Schueler-Furman, Sub-angstrom modeling of complexes between flexible peptides and globular proteins. Proteins 78, 2029-2040 (2010).

6. Quercetin Docking

Quercetin was docked to the PTPRD phosphatase using a two-step process of global docking followed by extra-precision local docking. The model for PTPRD's phosphatase was prepared for docking by adding hydrogens, assigning protonation states and optimizing hydrogen bonds using the Schrodinger Protein Preparation Wizard. Quercetin was prepared using LigPrep to enumerate protonation and tautomerization states and to generate an initial 3D structure. Global docking used Autodock Vina, a grid that fully encompassed PTPRD and 1,000 independent docking runs, providing an exhaustiveness parameter of 16. All of the top 1,000 scoring poses was confined to three sites or the catalytically-active phosphatase site. Local docking was performed at the three sites using the Glide program/extra precision (XP) protocol. Docked models were inspected for interactions that could explain the observed structure activity relationships.

7. Compound Screening and Docking Analysis

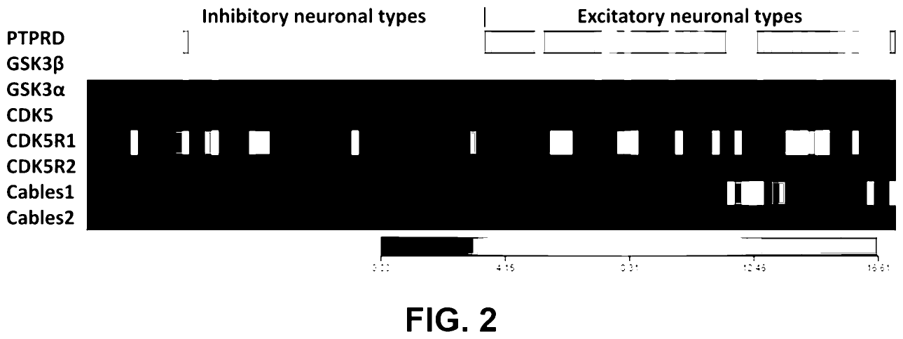

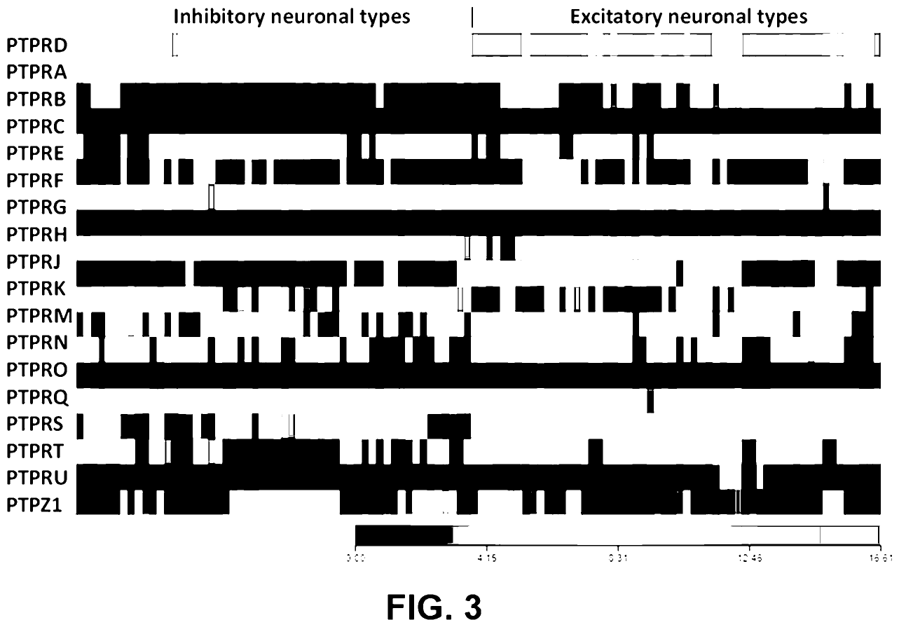

With reference to FIG. 1, the inventors have discovered that a conserved sequence that surround both pY279 in GSK3α and pY216 in GSK3β (SEQ ID NO: 2) and 2) a sequence that surrounds pY15 in CDK5 (SEQ ID NO: 4) are each good substrates for PTPRD's phosphatase (SEQ ID NO: 1). With references to FIGS. 2-3, PTPRD is positioned to interact with these three kinases because it is expressed more consistently and at higher levels than other receptor type protein tyrosine phosphatases in human cerebral cortical cell types including those that express GSK3β, GSK3α and/or CDK5 mRNAs, for example. It has also been discovered that heterozygous PTPRD knockout mice that provide models for common human PTPRD allelic variation display more phosphorylated pY216 GSK3β and pY279 GSK3α than wildtype littermates.

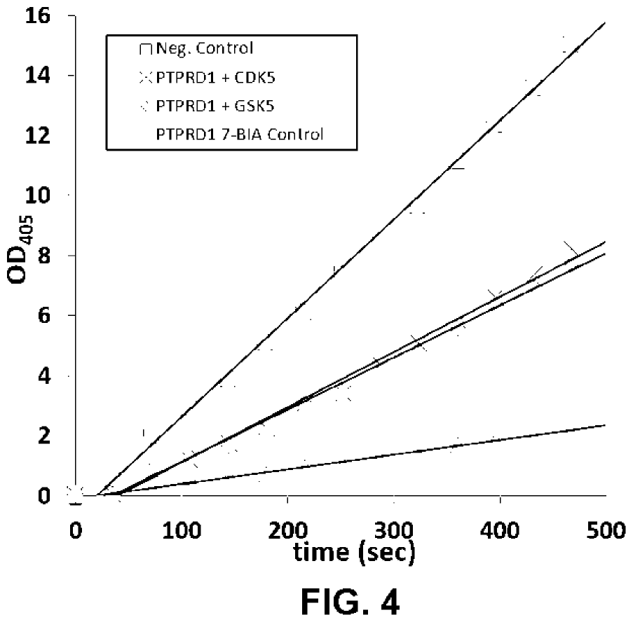

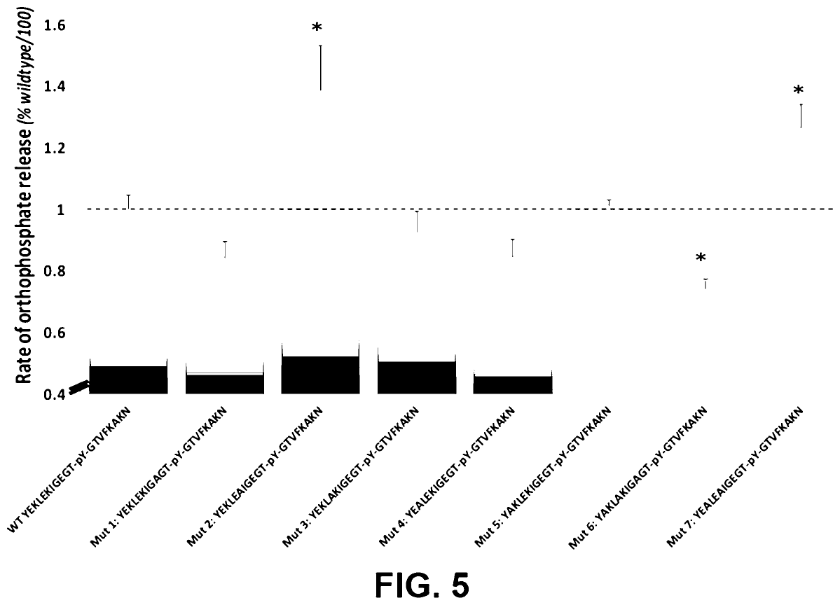

PTPRD's phosphatase liberates orthophosphate from CDK5 phosphopeptide at rates similar to those found for the generic positive control substrate END(pY)INASL (SEQ ID NO: 13); there is also substantial liberation of orthophosphate from GSK3 phosphopeptide. With reference to FIG. 4, each of these phosphopeptides also competes for PTPRD phosphatase's hydrolysis of the generic nonpeptide phosphatase substrate paranitrophenyl phosphate (pNPP). It was discovered that dephospho GSK3β, GSK3α and CDK5 peptides (SEQ ID NOs: 3 and 5, respectively) were inactive in competing for pNPP hydrolysis by the PTPRD phosphatase. Additionally, with reference to FIG. 5, mutant pY15CDK5 phosphopeptides (SEQ ID NOs: 6-12) display structure activity relationships that agree with data for random peptide sequences tested at PTPRD's phosphatase. Mutants with alanine substitutions for glutamic acid residues were less avidly dephosphorylated by PTPRD's phosphatase and those with substitutions for lysines in this region were more avidly dephosphorylated.

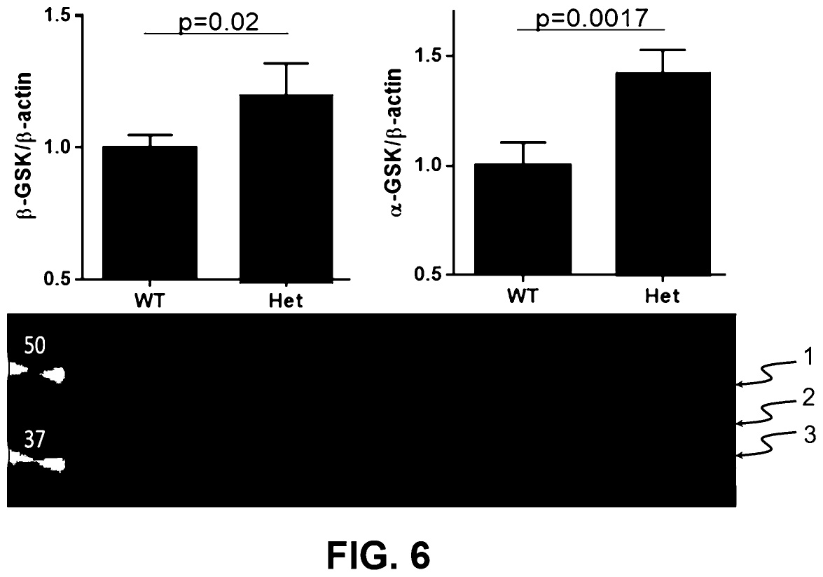

It was discovered that because PTPRD acts to regulate levels of tyrosine phosphorylation of these kinases in vivo, reducing levels of PTPRD can enhance brain levels of tyrosine-phosphorylated kinases. Accordingly, the inventors evaluated differences in pY216 GSK3β, pY279 GSK3α and pY15 CDK5 immunoreactivities in brains of heterozygous PTPRD knockout mice vs wildtype littermate control animals. With reference to FIG. 6, heterozygous knockout mouse brains displayed significantly-enhanced levels of both pY279 GSK3α and pY216 GSK3β vs wildtype littermates (increases were 1.29 and 1.15-fold (p=0.001 and 0.05) in combined data from FIG. 6 and a replicate experiment). By contrast, levels of pY15 CDK5 immunoreactivity displayed only nonsignificant trends toward lower levels of expression in brains of heterozygous knockouts.

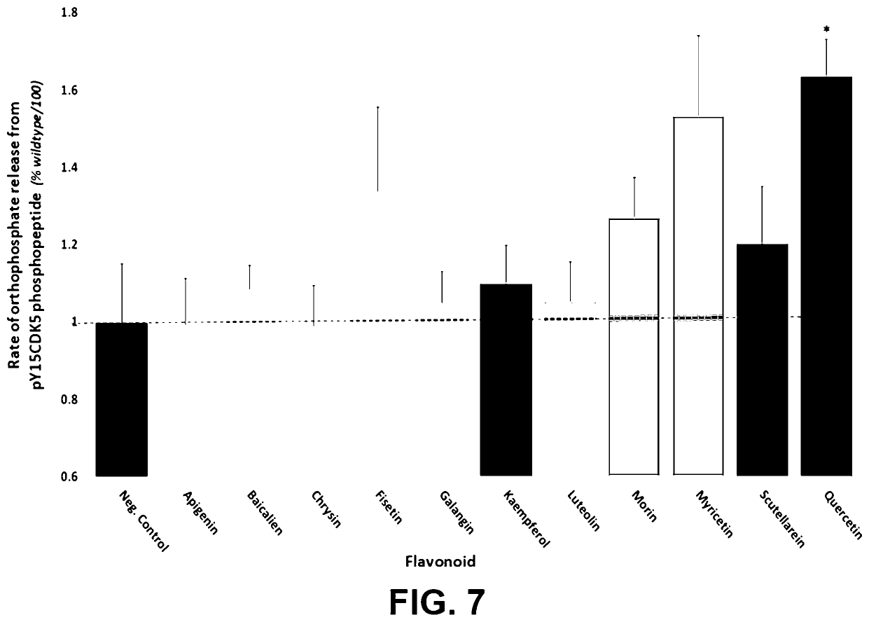

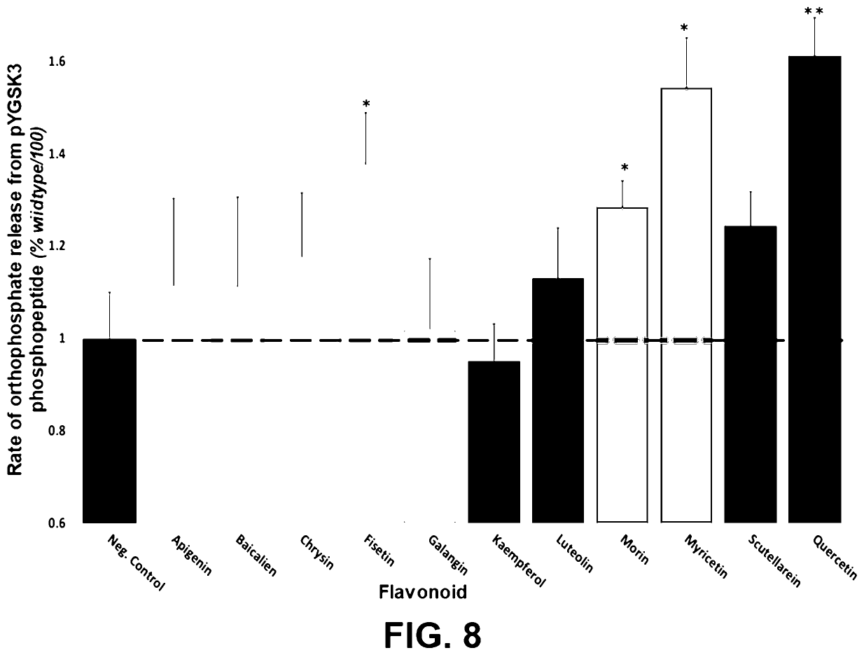

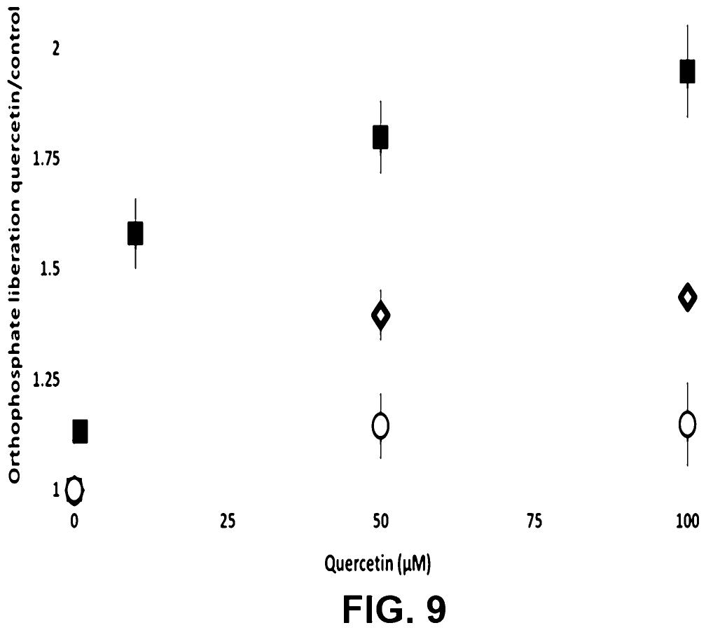

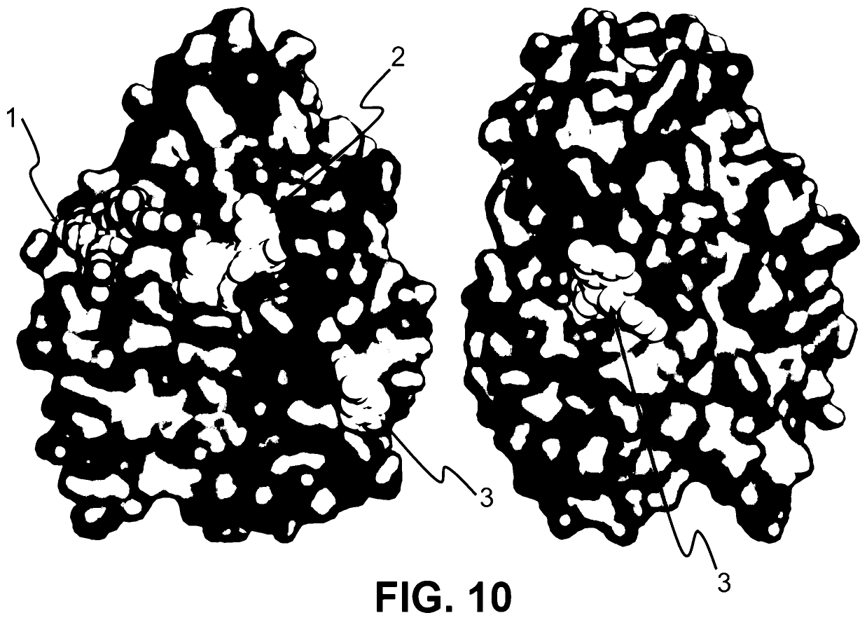

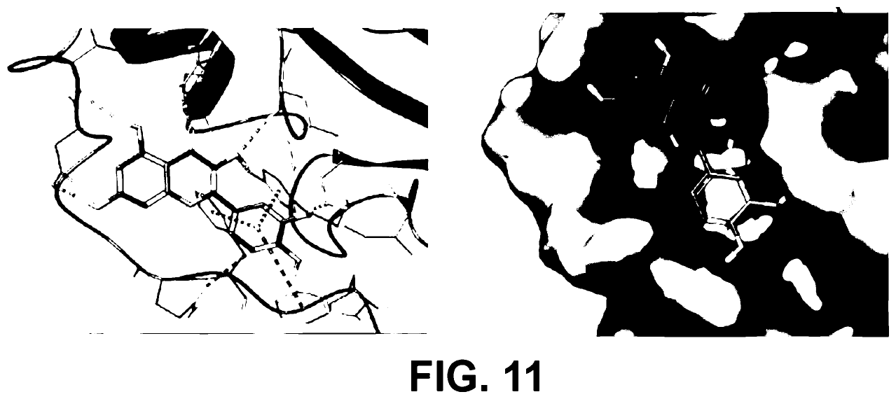

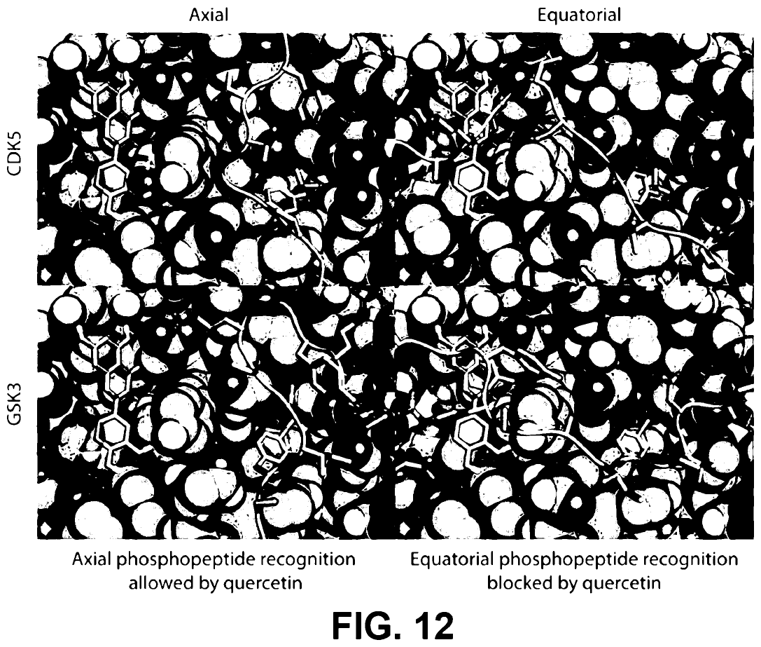

Various compounds were screened for activities at PTPRD's phosphatase. Surprisingly, quercetin and related compounds increased PTPRD's activity in dephosphorylating GSK3 and CDK5 phosphopeptides (see FIG. 1 and FIGS. 7-8). With reference to FIG. 9, quercetin was active at micromolar concentrations. There was significant (GSK3) and trend-level (CDK5) positive allosteric modulation for the related flavanols myricetin, fisetin and morin. In silico docking results support specific interactions between PTPRD's phosphatase, quercetin and GSK3 and CDK5 phosphopeptides. Docking quercetin to PTPRD's phosphatase identifies a binding site (FIGS. 10-12) that provides good −6.2 kcal/mol calculated binding energy. Quercetin's 3-hydroxyl contributes two hydrogen bonds.

GSK3 and CDK5 phosphopeptides can dock in silico with the PTPRD phosphatase in both “equatorial” and “axial” modes (520 vs 507 Rosetta energy units for GSK3 and 520 vs 539 for CDK5 for these arbitrarily-defined modes, respectively). Without wishing to be bound by any theory, the greater preference for equatorial binding of GSK3 vs CDK5 phosphopeptides fits with the inventors' observations (e.g., rates of PTPRD dephosphorylation of pYGSK3 are less than those for pYCDK5) if equatorial binding leads to less efficient dephosphorylation. “Equatorial” phosphopeptide binding to the PTP1C phosphatase leaves its catalytically-important WPD loop in an open, likely less active, conformation.

It will be apparent to those skilled in the art that various modifications and variations can be made without departing from the scope or spirit of this disclosure. Other embodiments will be apparent to those skilled in the art from consideration of the specification and practice disclosed herein. It is intended that the specification and examples be considered as exemplary only, with a true scope and spirit being indicated by the following claims.

What is claimed is:

1. A method of treating a tauopathy or restless leg syndrome in a subject, comprising administering to the subject an effective amount of a compound represented by Formula (I): 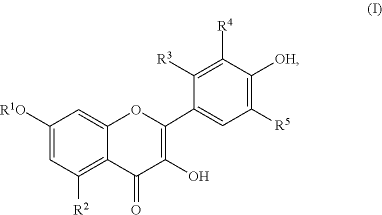
or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

2. The method of claim 1, wherein the compound is:

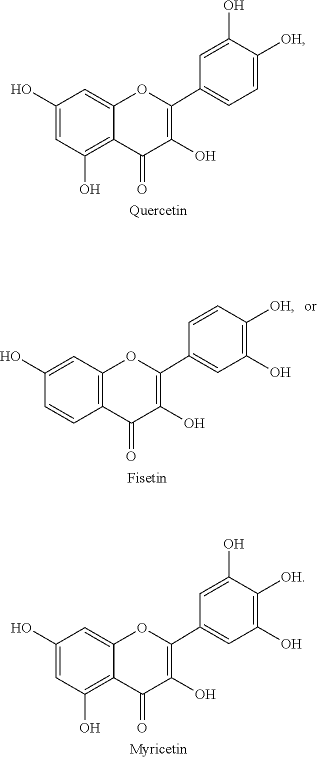 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

3. The method of claim 1, wherein the compound is Quercetin.

4. The method of claim 1, wherein the tauopathy is Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, or progressive supranuclear palsy.

5. The method of claim 1, further comprising administering to the subject an effective amount of a compound known for treating the tauopathy or restless leg syndrome.

6. A method of enhancing the ability of receptor-type tyrosine-protein phosphatase delta (PTPRD) to dephosphorylate a kinase, the method comprising contacting PTPRD with an effective amount of a compound represented by Formula (I): 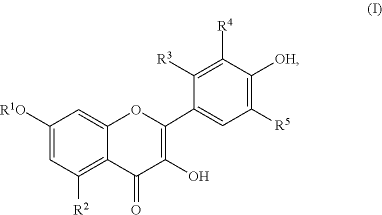
or a pharmaceutically acceptable salt thereof; wherein R¹ is hydrogen or —CH₃; R², R³, and R⁵ are each independently hydrogen or —OH; and R⁴ is hydrogen, —OH, or —OCH₃.

7. The method of claim 6, wherein the compound is:

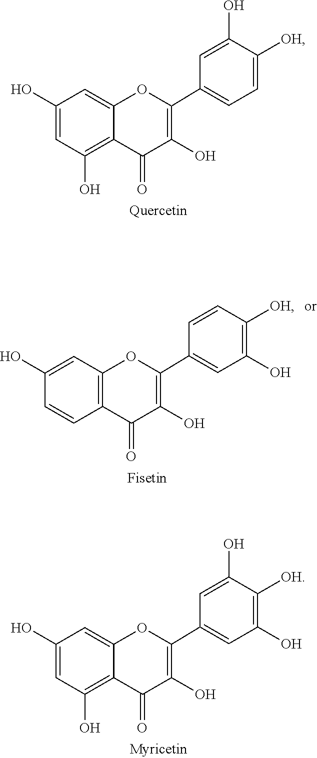 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

8. The method of claim 6, wherein the compound is Quercetin.

9. The method of claim 6, wherein the kinase is glycogen synthase kinase GSK3β, glycogen synthase kinase GSK3α, cyclin dependent kinase-5 CDK5, or a combination thereof.

10. A kit comprising:
a) a compound represented by Formula (I) in an amount effective for treating a tauopathy or restless leg syndrome in a subject, 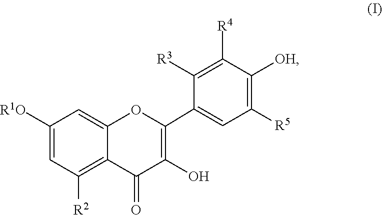
or a pharmaceutically acceptable salt thereof; wherein R¹ is hydrogen or —CH₃; R², R³, and R⁵ are each independently hydrogen or —OH; and R⁴ is hydrogen, —OH, or —OCH₃; and
b) instructions for treating the tauopathy or restless leg syndrome and/or an effective amount of a compound known for treating the tauopathy or restless leg syndrome.

11. The kit of claim 10, wherein the compound represented by Formula (I) is:

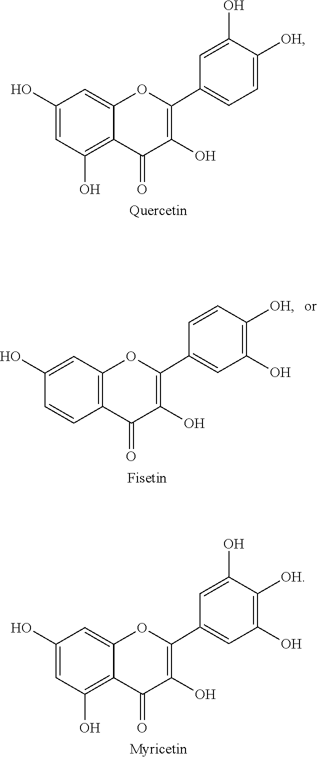 [SMILES: O=c1c(O)c(-c2ccc(O)c(O)c2)oc2cc(O)cc(O)c12 ; O=c1c(O)c(-c2ccc(O)c(O)c2)oc2cc(O)ccc12 ; O=c1c(O)c(-c2cc(O)c(O)c(O)c2)oc2cc(O)cc(O)c12]

12. The kit of claim 10, wherein the compound represented by Formula (I) is Quercetin.

13. The kit of claim 10, wherein the tauopathy is Alzheimer's disease, chronic traumatic encephalopathy, corticobasal degeneration, frontotemporal lobar degeneration, behavioral variant frontotemporal dementia, language variant frontotemporal dementia, right temporal variant frontotemporal dementia, Pick disease, or progressive supranuclear palsy.

14. The kit of claim 10, wherein the effective amount of the compound of Formula (I) and/or the compound known for treating the tauopathy or restless leg syndrome is a therapeutically effective amount.

15. The kit of claim 10, wherein the effective amount of the compound of Formula (I) and/or the compound known for treating the tauopathy or restless leg syndrome is a prophylactically effective amount.

16. A method of screening for positive allosteric modulators of the ability of a receptor-type tyrosine-protein phosphatase delta (PTPRD) to dephosphorylate a kinase, the method comprising:
a) contacting the PTPRD with a test compound in the presence of the phosphorylated kinase; and
b) measuring any orthophosphate release from the kinase;
wherein the PTPRD comprises a phosphatase D1 domain having at least 80% amino acid identity with SEQ ID NO: 1; and
wherein the kinase is GSKα or GSKβ comprising a polypeptide having at least 80% amino acid identity with SEQ ID NO: 2 or CDK5 comprising a polypeptide having at least 80% amino acid identity with SEQ ID NO: 4.

17. The method of claim 16, wherein the phosphatase D1 domain is SEQ ID NO: 1.

18. The method of claim 16, wherein the kinase is GSKα or GSKβ comprising a polypeptide having SEQ ID NO: 2 or CDK5 comprising a polypeptide having SEQ ID NO: 4.

19. The method of claim 16, wherein the test compound is a flavanoid.

20. The method of claim 16, wherein the test compound is a flavanol.


SEQUENCE LISTING (13 sequences)

SEQ ID NO: 1 - protein, 301 aa, Artificial Sequence
  Synthetic construct
MHHHHHHASHPPIPILELADHIERLKANDNLKFSQEYESIDPGQQFTWEHSNLEVNKPKNRYANVIAYDHSRVLLSAIEGIPGSDYVNANYIDGYRKQNAYIATQGSLPETFGDFWRMIWEQRSATVVMMTKLEERSRVKCDQYWPSRGTETHGLVQVTLLDTVELATYCVRTFALYKNGSSEKREVRQFQFTAWPDHGVPEHPTPFLAFLRRVKTCNPPDAGPMVVHCSAGVGRTGCFIVIDAMLERIKHEKTVDIYGHVTLMRAQRNYMVQTEDQYIFIHDALLEAVTCGNTEVPARNL

SEQ ID NO: 2 - protein, 22 aa, Artificial Sequence
  Synthetic construct
  MISC_FEATURE (11)..(11): phosphorylated tyrosine
QLVRGEPNVSPYICSRYYRAPE

SEQ ID NO: 3 - protein, 22 aa, Artificial Sequence
  Synthetic construct
QLVRGEPNVSSYICSRYYRAPE

SEQ ID NO: 4 - protein, 21 aa, Artificial Sequence
  Synthetic construct
  MISC_FEATURE (12)..(12): phosphorylated tyrosine
YEKLEKIGEGTPYGTVFKAKN

SEQ ID NO: 5 - protein, 20 aa, Artificial Sequence
  Synthetic construct
YEKLEKIGEGTYGTVFKAKN

SEQ ID NO: 6 - protein, 21 aa, Artificial Sequence
  Synthetic construct
  MISC_FEATURE (12)..(12): phosphorylated tyrosine
YEKLEKIGAGTPYGTVFKAKN

SEQ ID NO: 7 - protein, 21 aa, Artificial Sequence
  Synthetic construct
  MISC_FEATURE (12)..(12): phosphorylated tyrosine
YEKLEAIGEGTPYGTVFKAKN

SEQ ID NO: 8 - protein, 21 aa, Artificial Sequence
  Synthetic construct
  MISC_FEATURE (12)..(12): phosphorylated tyrosine
YEKLAKIGEGTPYGTVFKAKN

SEQ ID NO: 9 - protein, 21 aa, Artificial Sequence
  Synthetic construct
  MISC_FEATURE (12)..(12): phosphorylated tyrosine
YEALEKIGEGTPYGTVFKAKN

SEQ ID NO: 10 - protein, 21 aa, Artificial Sequence
  Synthetic construct
  MISC_FEATURE (12)..(12): phosphorylated tyrosine
YAKLEKIGEGTPYGTVFKAKN

SEQ ID NO: 11 - protein, 21 aa, Artificial Sequence
  Synthetic construct
  MISC_FEATURE (12)..(12): phosphorylated tyrosine
YAKLAKIGAGTPYGTVFKAKN

SEQ ID NO: 12 - protein, 21 aa, Artificial Sequence
  Synthetic construct
  MISC_FEATURE (12)..(12): phosphorylated tyrosine
YEALEAIGEGTPYGTVFKAKN

SEQ ID NO: 13 - protein, 10 aa, Artificial Sequence
  Synthetic construct
  MISC_FEATURE (4)..(4): phosphorylated tyrosine
ENDPYINASL